(19) United States
(12) Patent Application Publication
(10) Pub. No.: US 2021/0145774 A1
(43) Pub. Date: May 20, 2021

(54) Treatment of alcoholic liver disease

(71) Applicant: CymaBay Therapeutics, Inc., Newark, CA (US)

(72) Inventor: Edward E. Cable, San Leandro, CA (US)

(73) Assignee: CymaBay Therapeutics, Inc., Newark, CA (US)

(21) Appl. No.: 17/096,217

(22) Filed: Nov. 12, 2020

Related U.S. Application Data
(60) Provisional application No. 62/935,439, filed on Nov. 14, 2019.

(51) Int. Cl. A61K 31/192; A61P 1/16

(52) U.S. Cl. CPC A61K 31/192; A61K 9/0053; A61P 1/16

(57) ABSTRACT

Treatment of alcoholic liver disease with seladelpar or a salt thereof.

### CROSS-REFERENCE TO RELATED APPLICATION

This application claims the benefit under 35 USC 119(e) of Application No. 62/935439, filed 14 Nov. 2019, the entire content of which is incorporated into this application by reference.

### FIELD OF THE INVENTION

This invention relates to the treatment of alcoholic liver disease.

### DESCRIPTION OF THE RELATED ART

The intestinal barrier

According to Assimakopoulos et al., “The Role of the Gut Barrier Function in Health and Disease”, Gastroenterol. Res., 11(4), 261-263 (2018) [internal citations omitted]: “The intestinal tract contains the body's largest interface between a person and his or her external environment. The complexity of its function is obvious when thinking that at the same time the intestine must serve two opposite functions; the selective permeability of needed nutrients from the intestinal lumen into the circulation and into the internal milieu in general and, on the other hand, the prevention of the penetration of harmful entities including microorganisms, luminal antigens, and luminal proinflammatory factors. The latter function is known as barrier function. The gut barrier function is comprised by three major lines of defence: 1) The biological barrier, which is made up of normal intestinal flora (gut microbiota) responsible for colonization resistance; 2) The immune barrier, which is composed of gut associated lymphoid tissue (GALT), effector and regulatory T cells, IgA producing B (plasma) cells, group 3 innate lymphoid cells, and, resident macrophages and dendritic cells in the lamina propria; and 3) The mechanical barrier, consisting of the closed-lining intestinal epithelial cells and by the capillary endothelial cells. . . . The term ‘bacterial translocation’ (BT), was first described by Berg and Garlington in 1979, as the phenomenon of passage of viable bacteria from the gastrointestinal tract through the epithelial mucosa into the lamina propria and then to the mesenteric lymph nodes and possibly other normally sterile organs. This initial definition was later widened to include the translocation of non-viable bacteria or their products, namely pathogen-associated molecular patterns (PAMPs), with main representative the intestinal endotoxin. BT occurs in healthy individuals in a low rate of 5-10%, serving two main physiological roles; the antigenic exposure of the gut immune system to be prepared for an effective immune response in case of extensive pathogen invasion, and the development of immune tolerance to several microbial antigens of commensal microflora.”

The intestinal barrier is also referred to as the intestinal epithelial barrier, the intestinal mucosal barrier, the gut vascular barrier, and the gut barrier, among other terms; and “intestinal barrier” includes any of these essentially synonymous terms. “Intestinal barrier dysfunction” refers to a decrease in function of the intestinal barrier, including essentially synonymous terms such as “intestinal barrier loss”, “gut vascular barrier disruption” and the like.

Alcoholic liver disease

Intestinal barrier dysfunction has been extensively associated with alcoholic liver disease (ALD, sometimes “alcoholic liver diseases”), a term that encompasses the liver manifestations of alcohol overconsumption, including fatty liver (hepatic steatosis), acute and chronic alcoholic hepatitis, and chronic hepatitis with liver fibrosis or cirrhosis. According to O'Shea et al., “Alcoholic liver disease: AASLD Practice Guidelines”, Hepatology. 51(1), 307-328 (2010), ALD is the major cause of liver disease in Western countries. Although steatosis (fatty liver) will develop in any individual who consumes a large quantity of alcoholic beverages over a long period of time, this process is transient and reversible. More than 90% of all heavy drinkers develop fatty liver whilst about 25% develop the more severe alcoholic hepatitis, and 15% cirrhosis. ALD is a worldwide problem, with an estimated mortality in 2002 of 150,000 per year, while in the United States, alcohol-related mortality was the third leading cause of death; and (according to OECD data) the United States had the highest national alcohol-related mortality at nearly 9000 deaths in 2018.

According to Groschwitz et al., “Intestinal barrier function: Molecular regulation and disease pathogenesis”, J. Allergy Clin. Immunol., 124(1), 3-20 (2009) [internal citations omitted]: “Experimental studies in rodents have also demonstrated that acute administration of alcohol induces mucosal damage in the upper small intestine, including villus ulceration, submucosal blebbing, and hemorrhagic erosions and intestinal barrier dysfunction. It is postulated that alcohol-induced intestinal permeability facilitates enhanced translocation of endotoxin to distant organs, leading to inflammation and tissue damage. Intragastric administration of endotoxin in the presence of alcohol to rodents led to significantly higher plasma endotoxin levels than animals fed endotoxin alone. Similar lesions have been found in healthy volunteers and active alcoholics following acute alcohol consumption, and plasma endotoxin levels in alcoholics were found to be 5-fold greater than in healthy control subjects. Although not fully understood, evidence suggests the mechanism underlying alcohol induced barrier dysfunction is related to the influx of inflammatory cells and release of various mediators, including cytokines, reactive oxygen species, leukotrienes, and histamine.”

Chen et al., “Dysbiosis-Induced Intestinal Inflammation Activates Tumor Necrosis Factor Receptor I and Mediates Alcoholic Liver Disease in Mice”, Hepatology, 61(3), 883-894 (2015) [internal citations omitted], note:

“Alcohol abuse is one of the leading causes of chronic liver disease (CLD) and liver-related deaths worldwide. A prominent feature of alcohol abuse is disruption of intestinal barrier function. Increased intestinal permeability is present in preclinical animal models and in patients with alcohol abuse. Microbial products, such as lipopolysaccharide (LPS), translocate from the intestinal lumen to the extraintestinal space, blood, and liver. In the liver, bacterial products induce inflammation and synergize with ethanol-induced hepatotoxicity to cause steatosis, steatohepatitis, and fibrosis.”

Chronic ethanol intake, such as seen in alcoholic liver disease, also disrupts bile acid metabolism by increasing the total bile acid pool and secondary bile acids.

Treatments for alcoholic liver disease

Suk et al., “Alcoholic liver disease: Treatment”, World J. Gastroenterol., 20(36), 12934-12944 (2014), summarize treatments for alcoholic liver disease as of the time of their writing. They note that “Immediate abstinence is the most important treatment option for patients with ALD” and that alcoholic steatosis can be reversed after abstinence for several weeks, and mention the use of agents such as baclofen, acamprosate, and naltrexone, and psychotherapy, as aids to encouraging abstinence. They also suggest nutritional therapy, though a Cochrane review by Koretz et al., “Nutritional support for liver disease”, The Cochrane Database of Systemic Reviews, 5(5), 1465-1858 (2012), https://doi.org//10.1002/14651858.CD008344.pub2, found that evidence did not support supplemental nutrition in liver disease; and treatments for alcohol withdrawal syndrome, such as benzodiazepines.

However, Suk et al. mention few treatments for the actual effects of alcoholic liver disease, such as alcoholic hepatitis. Corticosteroids are suggested for severe alcoholic hepatitis, though they are recommended only when severe liver inflammation is present; and pentoxifylline is considered as an alternative to corticosteroids, especially if there are contradictions to corticosteroid therapy. Anti-tumor necrosis factor (TNF)-α agents, e.g. infliximab and etanercept, are now considered possibly harmful and are not recommended. Liver transplantation is considered the only definitive therapy for alcoholic cirrhosis.

There exists a need for effective and tolerable therapy for alcoholic liver disease at its various stages, and for both chronic and acute treatment.

Seladelpar

Seladelpar (International Nonproprietary Name—INN) has the chemical name [4-({(2R)-2-ethoxy-3-[4-(trifluoromethyl)phenoxy]propyl}sulfanyl)-2-methylphenoxy]acetic acid [IUPAC name from WHO Recommended INN: List 77], and the code number MBX-8025. Seladelpar and its synthesis, formulation, and use is disclosed in, for example, U.S. Pat. No. 7,301,050 (compound 15 in Table 1, Example M, claim 49), U.S. Pat. No. 7,635,718 (compound 15 in Table 1, Example M), and U.S. Pat. No. 8106095 (compound 15 in Table 1, Example M, claim 14). Lysine (L-lysine) salts of seladelpar and related compounds are disclosed in US Patent No. 7709682 (seladelpar L-lysine salt throughout the Examples, crystalline forms claimed).

Seladelpar is an orally active, potent (2 nM) agonist of peroxisome proliferator-activated receptor-δ (PPARδ). It is specific (>600-fold and>2500-fold compared with PPARα and peroxisome proliferator-activated receptor-γreceptors). PPARδ activation stimulates fatty acid oxidation and utilization, improves plasma lipid and lipoprotein metabolism, glucose utilization, and mitochondrial respiration, and preserves stem cell homeostasis. According to U.S. Pat. No. 7,301,050, PPARδ agonists, such as seladelpar, are suggested to treat PPARδ-mediated conditions, including “diabetes, cardiovascular diseases, Metabolic X syndrome, hypercholesterolemia, hypo-high density lipoprotein (HDL)-cholesterolemia, hyper-low density lipoprotein (LDL)-cholesterolemia, dyslipidemia, atherosclerosis, and obesity”, with dyslipidemia said to include hypertriglyceridemia and mixed hyperlipidemia.

U.S. Pat. No. 9,486,428 and PCT International Publication No. WO 2015/143178 disclose the treatment of intrahepatic cholestatic diseases, such as primary biliary cholangitis, primary sclerosing cholangitis, progressive familial intrahepatic cholestasis, and Alagille syndrome, with seladelpar and its salts; U.S. Pat. No. 9,381,181 and PCT International Publication No. WO 2015/157697 disclose the treatment of non-alcoholic fatty liver disease and non-alcoholic steatohepatitis with seladelpar and its salts; US Application Publication No. 2015-0374649 and PCT International Publication No. WO 2015/200580 disclose the treatment of severe hypertriglyceridemia with seladelpar and its salts; and US Application Publication No. 2015-0139987 and PCT International Publication No. WO 2015/077154 disclose the treatment of homozygous familial hypercholesterolemia with seladelpar and its salts.

Seladelpar has also been studied in primary biliary cholangitis (PBC), with results for 50 and 200 mg/day reported in Jones et al., “Seladelpar (MBX-8025), a selective PPAR-δ agonist, in patients with primary biliary cholangitis with an inadequate response to ursodeoxycholic acid: a double-blind, randomised, placebo-controlled, phase 2, proof-of-concept study”, Lancet Gastroenterol. Hepatol., 2(10), 716-726 (2017), and for 2, 5, and 10 mg/day at The International Liver Congress™ hosted by the European Association for the Study of Liver Diseases (EASL) in Paris, France (April 11-15, 2018): in poster LBP-2 (Hirschfield et al., “Treatment Efficacy and Safety of Seladelpar, a Selective Peroxisome Proliferator-Activated Receptor Delta agonist, in Primary Biliary Cholangitis Patients: 12- and 26-Week Analyses of an Ongoing, International, Randomized, Dose Ranging Phase 2 Study”), and in poster THU-239 (Boudes et al., “Seladelpar's Mechanism of Action as a Potential Treatment for Primary Biliary Cholangitis and Non-Alcoholic Steatohepatitis”), both available at https://ir.cymabay.com/presentations.

Haczeyni et al., “The Selective Peroxisome Proliferator-Activated Receptor-Delta Agonist Seladelpar Reverses Nonalcoholic Steatohepatitis Pathology by Abrogating Lipotoxicity in Diabetic Obese Mice”, Hepatol. Comm., 1(7), 663-674 (2017), have reported that seladelpar improves NASH pathology (reducing hepatic steatosis and inflammation, and improving fibrosis) in atherogenic diet-fed obese diabetic (Alms1 mutant (foz/foz)) mice, a well-known animal model for human NAFLD/NASH. Choi et al., “Seladelpar Improves Hepatic Steatohepatitis and Fibrosis in a Diet-Induced and Biopsy-Confirmed Mouse Model of NASH”, Abstract 1311 for the Liver Meeting® 2018 of the American Association for the Study of Liver Diseases (AASLD), have reported similar results in atherogenic diet-fed normal mice (DIO-NASH). CymaBay Therapeutics has completed a Phase 2b study of seladelpar in patients with NASH using doses of 10, 20, and 50 mg/day, NCT03551522: see CymaBay press release “CymaBay Therapeutics Announces the Initiation of a Phase 2b Study of Seladelpar in Patients with Non-Alcoholic Steatohepatitis”, https://ir.cymabay.com/pres s-releases/detail/431/cymabay-therapeutics-announces-the-initiation-of-a-phase-2b-study-of-seladelpar-in-patients-with-non-alcoholic-steatohepatitis.

The entire disclosures of the documents referred to in this application are incorporated into this application by reference.

### SUMMARY OF THE INVENTION

This invention is the treatment of alcoholic liver disease by administration of seladelpar or a salt thereof.

In view of the demonstrated efficacy of seladelpar in stabilizing intestinal barrier function; reducing serum alanine aminotransferase (ALT), hepatic triglycerides (TGs), and total bile acids; reducing hepatic steatosis and stainable lipids; and restoring normal hepatic architecture; in a mouse model of alcoholic liver disease, seladelpar is expected to have activity in treating alcoholic liver disease.

Preferred aspects of this invention are characterized by the specification and by the features of claims 1 to 20 of this application as filed.

### DETAILED DESCRIPTION OF THE INVENTION

Definitions

The intestinal barrier, alcoholic liver disease, and treatments for alcoholic liver disease are described in the subsections entitled “The intestinal barrier”, “Alcoholic liver disease”, and

“Treatments for alcoholic liver disease” of the DESCRIPTION OF THE RELATED ART.

“Treating” or “treatment” of alcoholic liver disease includes one or more of:
(1) preventing or reducing the risk of the manifestations of alcoholic liver disease, or reducing the risk of consequences of those manifestations, such as reducing the risk of hepatic cirrhosis; i.e., causing the manifestations of alcoholic liver disease, or the consequences such as hepatic cirrhosis, not to develop in a subject who may be suffering from alcoholic liver disease but who does not yet experience or display the manifestations of that condition (i.e. prophylaxis);
(2) inhibiting the manifestations of alcoholic liver disease, i.e., arresting or reducing the development of the manifestations; and
(3) relieving the manifestations of alcoholic liver disease, i.e., reducing the number, frequency, duration or severity of the manifestations.

A “therapeutically effective amount” of seladelpar or a seladelpar salt means that amount which, when administered to a human for treating alcoholic liver disease, is sufficient to effect treatment for the disease. The therapeutically effective amount for a particular subject varies depending upon the age, health and physical condition of the subject to be treated, the disease and its extent, the assessment of the medical situation, and other relevant factors. It is expected that the therapeutically effective amount will fall in a relatively broad range that can be determined through routine trial.

Seladelpar is described in the subsection entitled “Seladelpar” of the DESCRIPTION OF THE RLEATED ART.

Salts (for example, pharmaceutically acceptable salts) of seladelpar are included in this invention and are useful in the methods described in this application. These salts are preferably formed with pharmaceutically acceptable acids. See, for example, “Handbook of Pharmaceutically Acceptable Salts”, Stahl and Wermuth, eds., Verlag Helvetica Chimica Acta, Zurich, Switzerland, for an extensive discussion of pharmaceutical salts, their selection, preparation, and use. Unless the context requires otherwise, reference to seladelpar is a reference both to the compound and to its salts.

Because seladelpar contains a carboxyl group, it may form salts when the acidic proton present reacts with inorganic or organic bases. Typically, seladelpar is treated with an excess of an alkaline reagent, such as hydroxide, carbonate or alkoxide, containing an appropriate cation. Cations such as Na⁺, K⁺, Ca²⁺, Mg²⁺, and NH₄⁺ are examples of cations present in pharmaceutically acceptable salts. Suitable inorganic bases, therefore, include calcium hydroxide, potassium hydroxide, sodium carbonate and sodium hydroxide. Salts may also be prepared using organic bases, such as salts of primary, secondary and tertiary amines, substituted amines including naturally-occurring substituted amines, and cyclic amines, including isopropylamine, trimethylamine, diethylamine, triethylamine, tripropylamine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, N-ethylpiperidine, and the like. Useful salts are expected to include the L-lysine salts; and, as noted in the “Seladelpar” subsection, seladelpar is currently formulated as its L-lysine dihydrate salt.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”; and, for example, a formulation “comprising” a compound must contain that compound but also may contain other active ingredients and/or excipients.

Formulation and Administration

Seladelpar may be administered by any route suitable to the subject being treated and the nature of the subject's condition. Routes of administration include administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in “Remington: The Science and Practice of Pharmacy”, 20th ed., Gennaro, ed., Lippincott Williams & Wilkins, Philadelphia, Pa., U.S.A. Because seladelpar is orally available, typical formulations will be oral, and typical dosage forms will be tablets or capsules for oral administration. As mentioned in the “Seladelpar” subsection, seladelpar has been formulated in capsules for clinical trials. Intravenous formulations may be particularly applicable for administration to acutely ill subjects, such as subjects suffering from acute alcoholic hepatitis or alcoholic fibrosis or cirrhosis, such as those subjects who may be hospitalized for treatment.

Depending on the intended mode of administration, the pharmaceutical compositions may be in the form of solid, semi-solid or liquid dosage forms, preferably in unit dosage form suitable for single administration of a precise dosage. In addition to an effective amount of seladelpar, the compositions may contain suitable pharmaceutically-acceptable excipients, including adjuvants which facilitate processing of the active compounds into preparations which can be used pharmaceutically. “Pharmaceutically acceptable excipient” refers to an excipient or mixture of excipients which does not interfere with the effectiveness of the biological activity of the active compound(s) and which is not toxic or otherwise undesirable to the subject to which it is administered.

For solid compositions, conventional excipients include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in water or an aqueous excipient, such as, for example, water, saline, aqueous dextrose, and the like, to form a solution or suspension. If desired, the pharmaceutical composition to be administered may also contain minor amounts of nontoxic auxiliary excipients such as wetting or emulsifying agents, pH buffering agents and the like, for example, sodium acetate, sorbitan monolaurate, triethanolamine sodium acetate, triethanolamine oleate, etc.

For oral administration, the composition will generally take the form of a tablet or capsule; or, especially for pediatric use, it may be an aqueous or nonaqueous solution, suspension or syrup. Tablets and capsules are preferred oral administration forms. Tablets and capsules for oral use will generally include one or more commonly used excipients such as lactose and corn starch. Lubricating agents, such as magnesium stearate, are also typically added. When liquid suspensions are used, the active agent may be combined with emulsifying and suspending excipients. If desired, flavoring, coloring and/or sweetening agents may be added as well. Other optional excipients for incorporation into an oral formulation include preservatives, suspending agents, thickening agents, and the like.

Typically, a pharmaceutical composition of seladelpar, or a kit comprising compositions of seladelpar, is packaged in a container with a label, or instructions, or both, indicating use of the pharmaceutical composition or kit in the treatment of alcoholic liver disease.

A person of ordinary skill in the art of pharmaceutical formulation will be able to prepare suitable pharmaceutical compositions of the seladelpar by choosing suitable dosage forms, excipients, packaging, and the like, to achieve therapeutically effective formulations without undue experimentation and in reliance upon personal knowledge and the disclosure of this application.

A suitable amount of seladelpar or a salt thereof for oral dosing, for an adult subject with alcoholic liver disease, depending on the extent and severity of the disease, and factors such as hepatic and renal function, is expected to be between 1 and 200 mg/day, preferably between 5 and 100 mg/day, such as 5, 10, 20, 50, or 100 mg/day, when the amount is calculated as seladelpar. “When the amount is calculated as seladelpar” means that if a seladelpar salt is being used, the amount of that salt will be the amount that is equivalent to the stated amount of seladelpar; for example, if seladelpar L-lysine dihydrate salt is being used, the amount will be multiplied by the formula weight of seladelpar L-lysine dihydrate salt divided by the formula weight of seladelpar, or about 1.41; so that an amount of 100 mg/day when the amount is calculated as seladelpar will require an amount of about 141 mg/day of seladelpar L-lysine dihydrate salt. That is, a suitable amount of seladelpar for oral dosing is expected to be similar to the amounts employed in clinical trials for PBC, PSC, NASH, and other conditions. Suitable reductions in dose toward or below the lower end of the outer range above will be made for subjects who are children, depending on such additional factors as age and body mass; and in subjects with significant hepatic impairment, such as subjects in Child-Pugh classes B and C, depending on the degree of impairment. These amounts represent an average daily dose, and not necessarily an amount given at a single dose. Dosing may be as frequent as more than once/day (where the amount, or daily dose, will be divided between the number of administrations per day), but will more typically be once/day (where the amount is given in a single administration). Optionally, particularly in cases of significant hepatic impairment, the dosing may be less frequent than once/day, such as between once/week and every other day, for example once/week, twice/week (especially with the doses at least three days apart), three times/week (especially with the doses at least two days apart), or every other day. Similar amounts and dosing schedules are expected to be applicable for dosing by injection, such as for intravenous administration.

A person of ordinary skill in the art of the treatment of alcoholic liver disease, who will typically be a person of ordinary skill in the art of the treatment of intestinal and hepatobiliary diseases, will be able to ascertain a therapeutically effective amount of seladelpar for a particular extent of disease and patient to achieve a therapeutically effective amount for the treatment of alcoholic liver disease without undue experimentation and in reliance upon personal knowledge, the skill of the art, and the disclosure of this application.

### EXAMPLES

### Example 1

### Preclinical, Lieber-DeCarli ethanol diet

Age-matched wild-type female C57BL/6 mice (Charles River, Wilmington, Mass.), 8-9 weeks old at the initiation of the study, were used. The mice were divided into five study groups with initial numbers as follows:
(1) control diet (n=10);
(2) control diet with seladelpar prevention (n=10);
(3) ethanol diet (n=24);
(4) ethanol diet with seladelpar prevention (n=24); and
(5) ethanol diet with seladelpar intervention (n=24).

The Lieber-DeCarli ethanol diet was originally developed by Charles Lieber and Leonore DeCarli in 1963. This diet allows for the prolonged exposure of ethanol in a rodent model and allows for modification to calories provided by ethanol. The Lieber-DeCarli ethanol diet used consisted of PMI® Micro Stabilized Alcohol Rodent Liquid Diet LD101A and PMI® Maltodextrin LD104, both from TestDiet (St. Louis, Mo.) and 200 proof ethanol from Gold Shield (Hayward, Calif.) in a specific combination following TestDiet's preparation and feeding directions [https://www.testdiet.com/cs/-groups/lolweb/@testdiet/documents/web_content/mdrf/mdi2/˜edisp/ducm04_026403.pdf]. The caloric intake from ethanol was 0% on day 1, 10% on days 2 and 3, 20% on days 4 and 5, 30% from day 6 until the end of 6 weeks, and 36% for the last 2 weeks, for a total in-life time of eight weeks. The ethanol-fed mice also received one bolus dose of 33% v/v ethanol at 5 g/Kg on the last in-life day. The control diet contained an isocaloric substitution of isomaltose for the ethanol. Where appropriate, seladelpar (as a solution made from the L-lysine dihydrate salt) was added at 0.015 mg seladelpar/mL to the prepared liquid diet: assuming a consumption of about 0.68 mL/day, this resulted in an oral dose of seladelpar of 10 mg/Kg/day. In the prevention portions of the study, including with the control diet, seladelpar was added throughout the entire study period; in the intervention portion of the study, seladelpar was added only for the fifth through eighth weeks. Fresh feces were collected at 7.5 weeks. All mice were sacrificed after eight weeks; the ethanol-fed mice eight hours after the ethanol bolus. At sacrifice, body and liver weight were recorded; and blood/plasma, liver, gallbladder, small and large intestine (wall and contents) and cecum were harvested. Analyses included plasma ALT—to measure inflammation; hepatic TGs; plasma lipopolysaccharides (LPS)—to measure the extent of bacterial leakage from the intestine into the blood; fecal albumin (FA)—to measure the extent of albumin leakage from the blood into the intestine (feces); and total bile acids, measured by liquid chromatography-mass spectrometry (LC-MS) generally according to the method described in Hartmann et al., “Modulation of the Intestinal Bile Acid/Farnesoid X Receptor/Fibroblast Growth Factor 15 Axis Improves Alcoholic Liver Disease in Mice”, Hepatology, 67(6), 2150-2166 (2018). Plasma ethanol was also measured in the ethanol group, and did not vary significantly between groups 3, 4, and 5.

The study results are given in the table below: standard errors of the means are in parentheses, n denotes the number of mice measured, and ANOVA denotes the significance value of an ANOVA analysis between groups 3, 4, and 5:

| Study group | 1 | 2 | 3 | 4 | 5 | ANOVA |
|---|---|---|---|---|---|---|
| ALT (U/L) | 17.9 (5.7) | 20.8 (3.5) | 83.3 (16.7) | 37.8 (4.1) | 30.9 (4.1) | <0.001 |
| | n = 10 | n = 10 | n = 19 | n = 24 | n = 22 | |
| Hepatic TGs | 21.7 (1.5) | 18.9 (1.8) | 29.6 (2.0) | 21.8 (1.3) | 20.5 (1.3) | <0.001 |
| (mg/g) | n = 10 | n = 10 | n = 19 | n = 24 | n = 22 | |
| Plasma LPS | 645 (93) | 787 (108) | 1064 (204) | 723 (66) | 634 (59) | 0.034 |
| (ng/mL) | n = 10 | n = 10 | n = 18 | n = 23 | n = 22 | |
| FA (ng/mg) | 104 (12) | 95 (12) | 135 (15) | 98 (8) | 112 (9) | 0.060 |
| | n = 10 | n = 10 | n = 19 | n = 21 | n = 22 | |
| Total bile | 17.8 (1.6) | 14.2 (0.6) | 23.7 (1.6) | 16.5 (1.1) | 16.0 (1.1) | <0.001 |
| acids | n = 10 | n = 10 | n = 19 | n = 21 | n = 22 | |
| (μmol/100 g | | | | | | |
| body weight) | | | | | | |

Seladelpar prevention does not significantly affect any of the above parameters in the control diet groups; but ANOVA analysis shows significant reductions (improvements) in serum ALT, hepatic TGs, serum LPS, and total bile acids between the untreated group 3 and the seladelpar-treated groups 4 and 5 (i.e., both the prevention and intervention groups) in the alcohol diet groups. Although ANOVA analysis of fecal albumin is not significant at the p<0.05 level, there is a significant reduction (improvement) in fecal albumin between the untreated group 3 and the seladelpar-treated groups 4 and 5.

Formalin-fixed liver sections were stained for histological examination with hematoxylin and eosin (H+E). Examination of representative sections showed no significant change on seladelpar treatment for mice on the control diet (comparing groups 1 and 2); however, significant reduction of steatosis and restoration of a more normal architecture was seen on seladelpar treatment (both prevention and intervention) in the alcohol diet groups (comparing group 3 with groups 4 and 5).

Frozen liver sections were cut and stained with oil red 0 for hepatic lipid accumulation analysis. Examination of representative sections showed no significant change on seladelpar treatment for mice on the control diet (comparing groups 1 and 2); however, consistent with the observed reduction in hepatic TGs, significant reduction of stainable lipid droplets was seen on seladelpar treatment (both prevention and intervention) in the alcohol diet groups (comparing group 3 with groups 4 and 5).

This study demonstrates the benefit of seladelpar, in either prevention or intervention mode, in the treatment of alcoholic liver disease in this predictive model.

### Example 2

### Clinical (Oral)

Subjects with diagnosed alcoholic liver disease (alcoholic fatty liver) are treated with seladelpar or a salt thereof, orally at 2 mg/day, 5 mg/day, 10 mg/day, 20 mg/day, 50 mg/day, or 100 mg/day, calculated as seladelpar, for 6 months. Subjects are permitted their usual other medications (e.g. antidiabetic medications such as metformin or sulfonamides) but not glitazones, PPAR agonists, OCA, or similar medications. The subjects are assessed before the study, and at intervals during the study, such as every 4 weeks during the study and 4 weeks after the last dose of the seladelpar therapy, for safety and pharmacodynamic evaluations.

MRIs of the subjects' livers are taken every 4 weeks during the study and 4 weeks after completion of seladelpar dosing to determine hepatic fat (MRI-PDFF, see Lee et al., “Estimating of hepatic fat amount using MRI proton density fat fraction in a real practice setting”, Medicine (Baltimore), 96(33), e7778 (2017)). At each visit, after a 12-hour fast, blood is drawn and urine collected; and a standard metabolic panel, complete blood count, and standard urinalysis are performed. Blood is analyzed for bilirubin, creatinine, prothrombin time (International Normalized Ratio—INR), TGs, and liver transaminases. The subjects also maintain health diaries, which are reviewed at each visit. The subjects show a dose-related improvement in their disease, as manifested by, for example, reductions in MRI-PDFF, TGs, and liver transaminases.

### Example 3

### Clinical (Intravenous)

Subjects with diagnosed alcoholic liver disease (acute alcoholic hepatitis) are treated with seladelpar or a salt thereof intravenously at 2 mg/day, 5 mg/day, 10 mg/day, 20 mg/day, 50 mg/day, or 100 mg/day, calculated as seladelpar, for up to 30 days. The subjects are assessed before the study, and at intervals during the study, such daily during the study and 4 weeks after the last dose of the seladelpar therapy, for safety and pharmacodynamic evaluations.

At each assessment blood is drawn; and a standard metabolic panel and complete blood count are performed. Blood is analyzed for bilirubin, creatinine, prothrombin time (International Normalized Ratio—INR), TGs, liver transaminases, high sensitivity C-reactive protein (hsCRP), interleukin-18, and TNF-α. The subjects show a dose-related improvement in their disease, as manifested by, for example, reduction in liver transaminases, hsCRP, interleukin-18, and TNF-a; and reduction in Maddrey's discriminant function and MELD (Model for End-stage Liver Disease) score.

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims.

1. A method of treating alcoholic liver disease by administering a therapeutically effective amount of seladelpar or a salt thereof.

2. The method of claim 1 where the seladelpar or a salt thereof is a seladelpar L-lysine salt.

3. The method of claim 2 where the seladelpar L-lysine salt is seladelpar L-lysine dihydrate salt.

4. The method of claim 1 where the seladelpar or a salt thereof is administered orally.

5. The method of claim 1 where the seladelpar or a salt thereof is administered intravenously.

6. The method of claim 1 where the amount of seladelpar or a salt thereof is between 1 mg/day and 100 mg/day, when the amount is calculated as seladelpar.

7. The method of claim 6 where the amount of seladelpar or a salt thereof is at least 2 mg/day.

8. The method of claim 6 where the amount of seladelpar or a salt thereof is not more than 50 mg/day.

9. The method of claim 6 where the amount of seladelpar or a salt thereof is 2 mg/day, 5 mg/day, 10 mg/day, 20 mg/day, 50 mg/day, or 100 mg/day.

10. The method of claim 9 where the amount of seladelpar or a salt thereof is 2 mg/day.

11. The method of claim 9 where the amount of seladelpar or a salt thereof is 5 mg/day.

12. The method of claim 9 where the amount of seladelpar or a salt thereof is 10 mg/day.

13. The method of claim 9 where the amount of seladelpar or a salt thereof is 20 mg/day.

14. The method of claim 9 where the amount of seladelpar or a salt thereof is 50 mg/day.

15. The method of claim 9 where the amount of seladelpar or a salt thereof is 100 mg/day.

16. The method of claim 1 where the seladelpar or a salt thereof is administered once/day.

17. The method of claim 1 where the seladelpar or a salt thereof is administered between once/week and every other day.

18. The method of claim 1 where the alcoholic liver disease is alcoholic fatty liver.

19. The method of claim 1 where the alcoholic liver disease is acute alcoholic hepatitis.

20. The method of claim 1 where the alcoholic liver disease is alcoholic cirrhosis.
